(19) United States
(12) Patent Application Publication
(10) Pub. No.: US 2021/0398448 A1
(43) Pub. Date: Dec. 23, 2021

(54) DEVICE FOR IMPROVING CHILDREN'S ATTENTION TO URINATION

(71) Applicant: Zhangzhou Go Win Lighting Co., Ltd, Zhangzhou (CN)

(72) Inventors: Xiongzhong LIN, Zhangzhou (CN); Yao LIU, Zhangzhou (CN)

(21) Appl. No.: 16/986,223

(22) Filed: Aug. 5, 2020

(30) Foreign Application Priority Data
Jun. 18, 2020 (CN) 202021139757.6

(51) Int. Cl. G09B 19/00; A61G 9/00; G09B 19/24; A61L 2/10

(52) U.S. Cl. CPC G09B 19/0076; A61G 9/006; G09B 19/24; A61L 2/10; A61L 2202/20; A61G 2203/78; A61G 2203/10; A61G 2200/14; A61L 2202/11; A61G 2203/30

(57) ABSTRACT

A device for improving children's attention to urination includes a streamlined shell; a light module including a liquid sensor and a control unit and a plurality of light sources, the liquid sensor and the control unit and the plurality of light sources are connected one-by-one, the liquid sensor and the light sources are mounted on the surface of the streamlined shell, the control unit is mounted inside the streamlined shell; a power supply module mounted in the streamlined shell and used for supplying power to the light module. It can effectively increase children's attention to urination, keep children focusing on urinating in the liquid draining tray for lighting up more light sources or making sound. It can effectively prevent children from spilling urine out of the potty.

### CROSS REFERENCE TO RELATED APPLICATIONS

This application claims all benefits accruing under 35 U.S.C. § 119 from China Patent Application No. 202021139757.6 filed on Jun. 18, 2020, in the State Intellectual Property Office of China, the disclosure of which is incorporated herein by reference in its entirety.

### TECHNICAL FIELD

The disclosure generally relates to the field of urinal device for children, and more particularly to a device for improving children's attention to urination.

### BACKGROUND

Children refer to human beings from the beginning of life to before adulthood. Before the age of 6, children are in an active stage which cannot integrate received information.

When a child urinates, the child tends to be distracted, unable to concentrate on the potty, and easily spills urine out of the potty.

The purpose of this disclosure is to design a device for improving children's attention to urination for the above existing technical problems.

### SUMMARY OF DISCLOSURE

In view of the problems existing in the prior art, the disclosure provides a device for improving children's attention to urination, and can effectively solve the problems existing in the prior art.

The technical scheme of the disclosure is:

A device for improving children's attention to urination, comprising:

a streamlined shell;
a light module including a liquid sensor and a control unit and a plurality of light sources, the liquid sensor and the control unit and the plurality of light sources are connected one-by-one, the liquid sensor and the light sources are mounted on the surface of the streamlined shell, the control unit is mounted inside the streamlined shell; and
a power supply module mounted in the streamlined shell and used for supplying power to the light module.

Further, the device further comprises a sound-generating module, which is electrically connected to the control unit and used for making sound.

Further, the sound-generating module is mounted at a top of the streamlined shell.

Further, a bottom end of the streamlined shell is provided with a liquid draining tray, and the liquid sensor is mounted at a bottom of the liquid draining tray.

Further, the bottom of the liquid draining tray is provided with an ultraviolet disinfection lamp.

Further, both sides of the top end of the streamlined shell are provided with a slot, and a handle is mounted between the slots.

Further, the liquid sensor is one of a capacitive liquid sensor, a resistive liquid sensor, an infrared sensor, and a humidity sensor.

Further, the streamlined shell includes a straight part and a first arc part and a second arc part, the straight part and the first arc part and the second arc part are connected one-by-one.

Further, length of the straight part is defined as D1, range of D1 is 4-8 cm; radius of the first arc part is defined as R1, range of R1 is 4-6 cm; radius of the second arc part is defined as R2, range of R2 is 5-8 cm.

Further, the bottom of the straight part hinges a back clip, the angle between the back clip and the straight part is defined as A, and an angle of A is adjustable from 45 to 135°.

The technical scheme has the following technical effects:

1) In the present disclosure, the surface of the streamlined shell is provided with a light module, when the liquid is poured onto the liquid sensor, the liquid sensor sends a control signal, the control unit lights up different numbers of the light sources according to the duration of the control signal. It can effectively increase children's attention to urination, keep children focused on urinating in the liquid draining tray for lighting up more light sources or making sound. It can effectively prevent from children spilling urine out of the potty.

2) In the present disclosure, the bottom of the straight part hinges a back clip, which can be conveniently clamped on the potty and adjust the angle.

3) The device is provided with an ultraviolet disinfection lamp, which as the function of disinfection and sterilization.

4) The streamlined shell including a straight part and a first arc part and a second arc part, through the special structural design of these parts, effectively makes the device adapted to the inner wall curve of most potty, and can be placed into the potty conveniently, prevents spilling out of urine due to the mismatching between the appearance of the device and the potty.

### BRIEF DESCRIPTION OF DRAWINGS

FIG. 1 is a structural diagram of the disclosure.

FIG. 2 is a functional framework of the disclosure.

FIG. 3 is a structural diagram of a streamlined shell of the disclosure.

### DETAILED DESCRIPTION

To facilitate the understanding of those skilled in the art, the structure of the disclosure is further described in detail in connection with the accompanying drawings:

### Embodiment 1

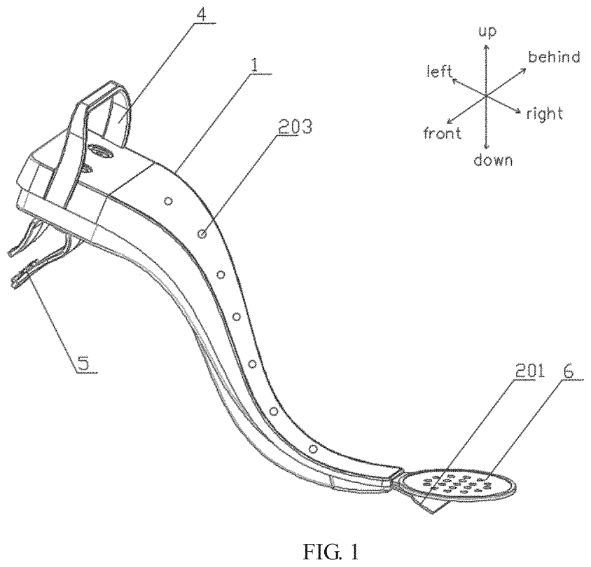

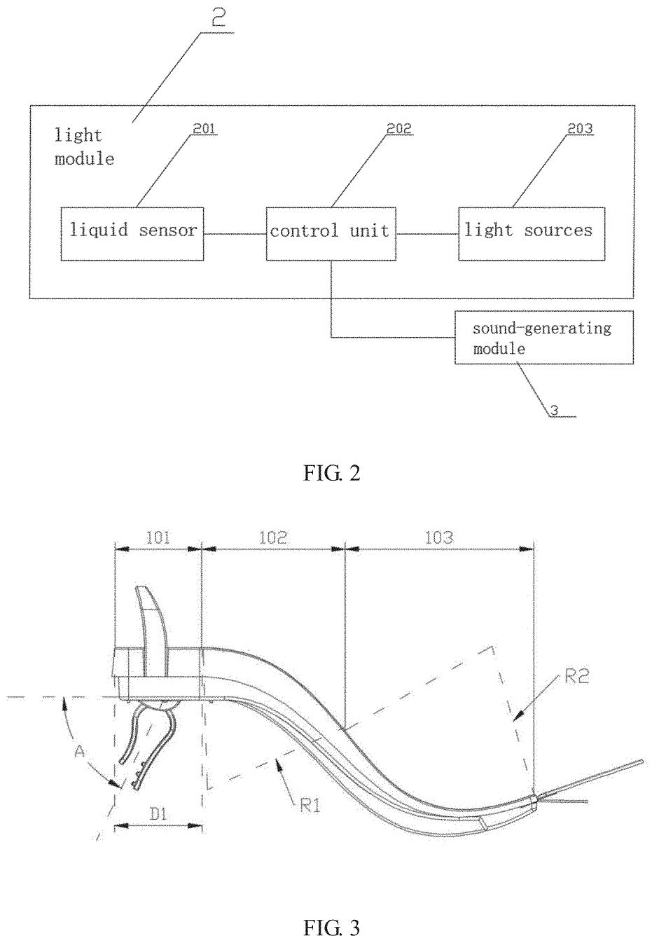

One embodiment of the disclosure, referring to FIGS. 1-3.

A device for improving children's attention to urination, comprising:

a streamlined shell 1;
a light module 2 including a liquid sensor 201 and a control unit 202 and a plurality of light sources 203, the liquid sensor 201 and the control unit 202 and the light sources 203 are connected one-by-one, the liquid sensor 201 and the plurality of light sources 203 are mounted on the surface of the streamlined shell 1, the control unit 202 is mounted inside the streamlined shell 1; the liquid sensor 201 is used to send a control signal when liquid is detected, and the liquid sensor 201 of this embodiment is inclined downward. When liquid drops on the liquid sensor 201, the liquid will quickly slide down from the liquid sensor 201, avoiding the misjudgment caused by liquid staying on the surface of the liquid sensor 201. The control unit 202 is used to light up different numbers of the light sources 203 according to the duration of the control signal; and
a power supply module mounted in the streamlined shell 1 and used for supplying power to the light module 2.

Specifically, the device further comprises a sound-generating module 3, which is electrically connected to the control unit 202 and used for making sound.

Specifically, the sound-generating module 3 is mounted at a top of the streamlined shell 1.

Specifically, wherein a bottom end of the streamlined shell 1 is provided with a draining tray 6, and the liquid sensor 201 is mounted at a bottom of the liquid draining tray 6.

Specifically, wherein the bottom of the liquid draining tray 6 is provided with an ultraviolet disinfection lamp.

Specifically, wherein both sides of the top end of the streamlined shell 1 are provided with a slot, and a handle 4 is mounted between the slots.

Specifically, wherein the liquid sensor 201 is one of a capacitive liquid sensor, a resistive liquid sensor, an infrared sensor, and a humidity sensor. The liquid sensor 201 specifically adopted in this embodiment is JR223B (commercially available) and belongs to a capacitive liquid sensor.

Specifically, the streamlined shell 1 includes a straight part 101 and a first arc 102 part and a second arc part 103, the straight part 101 and the first arc part 102 and the second arc part 103 are connected one-by-one.

Specifically, length of the straight part 101 is defined as D1, range of D1 is 4-8 cm; radius of the first arc part 102 is defined as R1, range of R1 is 4-6 cm; radius of the second arc part 103 is defined as R2, range of R2 is 5-8 cm. In this embodiment, D1 is 5 cm, R1 is 5 cm, R2 is 7 cm.

Specifically, the bottom of the straight part 101 hinges a back clip 5, the angle between the back clip 5 and the straight part 101 is defined as A, and an angle of A is adjustable from 45 to 135°.

In use, the device is placed at the potty and fixed the angle of the device through the back clip 5, so that the liquid sensor 201 is suspended in the center of the potty. When the liquid is poured onto the liquid sensor 201, the liquid sensor 201 sends a control signal, the control unit 202 lights up different numbers of the light sources 203 according to the duration of the control signal.

In this embodiment, the control signal lasts 1-5 seconds, the control unit 202 lights up one light sources 203 from the bottom, lasts 6-10 seconds, lights up two light sources 203 from the bottom, and so on.

In another embodiment, the control signal lasts for 1-5 seconds, the control unit 202 lights up one or two light sources 203, producing the effect that the first light source is always on and the second light source 203 blinks. The control signal lasts 6-10 seconds, the control unit 202 lights up two or three light sources, producing the effect that the first and second light sources are always on and the third light source 203 blinks, and so on.

The sound-generating module 3 is used for making sound according to the duration of the control signal. In this embodiment, when the duration of control signal is up 10 seconds, the sound-generating module plays music.

The streamlined shell 1 including a straight part 101 and a first arc part 102 and a second arc part 103, through the special structural design of these parts, effectively makes the present disclosure adapted to the inner wall curve of most potty, and can be placed into the potty conveniently, prevents spilling out of urine due to the mismatching between the appearance of the device and the potty.

The foregoing description is only a preferred embodiment of the disclosure, and all changes and modifications to the patent scope applied for in accordance with the disclosure shall belong to the scope covered by the disclosure.

What is claimed is:

1. A device for improving children's attention to urination, comprising:
a streamlined shell;
a light module including a liquid sensor and a control unit and a plurality of light sources, the liquid sensor and the control unit and the plurality of light sources are connected one-by-one, the liquid sensor and the light sources are mounted on the surface of the streamlined shell, the control unit is mounted inside the streamlined shell; and
a power supply module mounted in the streamlined shell and used for supplying power to the light module.

2. The device for improving children's attention to urination according to claim 1, wherein the device further comprises a sound-generating module, which is electrically connected to the control unit and used for making sound.

3. The device for improving children's attention to urination according to claim 2, wherein the sound-generating module is mounted at a top of the streamlined shell.

4. The device for improving children's attention to urination according to claim 1, wherein a bottom end of the streamlined shell is provided with a liquid draining tray, and the liquid sensor is mounted at a bottom of the liquid draining tray.

5. The device for improving children's attention to urination according to claim 4, wherein the bottom of the liquid draining tray is provided with an ultraviolet disinfection lamp.

6. The device for improving children's attention to urination according to claim 1, wherein both sides of the top end of the streamlined shell are provided with a slot, and a handle is mounted between the slots.

7. The device for improving children's attention to urination according to claim 1, wherein the liquid sensor is one of a capacitive liquid sensor, a resistive liquid sensor, an infrared sensor, and a humidity sensor.

8. The device for improving children's attention to urination according to claim 1, wherein the streamlined shell includes a straight part and a first arc part and a second arc part, the straight part and the first arc part and the second arc part are connected one-by-one.

9. The device for improving children's attention to urination according to claim 8,
wherein length of the straight part is defined as D1, range of D1 is 4-8 cm;
radius of the first arc part is defined as R1, range of R1 is 4-6 cm;
radius of the second arc part is defined as R2, range of R2 is 5-8 cm.

10. The device for improving children's attention to urination according to claim 8, wherein the bottom of the straight part hinges a back clip, the angle between the back clip and the straight part is defined as A, and an angle of A is adjustable from 45 to 135°.
